(19) United States
(12) Patent Application Publication
(10) Pub. No.: US 2007/0265168 A1
(43) Pub. Date: Nov. 15, 2007

(54) Moisture regulator

(75) Inventors: Anton Krallmann, Bad Fallingbostel (DE); Heinrich Henze-Wethkamp, Walsrode (DE)

Correspondence Address: CONNOLLY BOVE LODGE & HUTZ, LLP, P O BOX 2207, WILMINGTON, DE, 19899, US

(73) Assignee: Casetech GmbH & Co. KG, Walsrode (DE)

(21) Appl. No.: 11/789,390

(22) Filed: Apr. 24, 2007

(30) Foreign Application Priority Data
May 12, 2006 (DE) 102006022707.7

(51) Int. Cl. A01N 25/16; A01N 25/04; A61F 2/00

(52) U.S. Cl. 504/366; 424/423

(57) ABSTRACT

The present invention relates to a regulator for the targeted provision of moisture which is uniform with respect to time, for example in packaging or transport containers.

### CROSS-REFERENCE TO RELATED APPLICATION

The present application in entitled to the benefits of German Patent Application No. 10 2006 022 707.7 filed May 12, 2006, and that application is incorporated herein in its entirety for all useful purposes.

### BACKGROUND OF THE INVENTION

1. Field of the Invention

The present invention relates to a novel regulator for the targeted provision of moisture which is uniform with respect to time, for example in packaging or transport containers.

2. Related Art

Water-absorbing food casings such as artificial skins based on polyamide or cellulose are customarily provided in the form of what are termed shirred sticks for the meat-processing industry (DE 89 44 623 C2). On account of the significantly reduced length compared with the roll product, the processor can process the tubular casing considerably more economically. Such shirred sticks customarily have only a very low water content to no water content at all, and are therefore rigid and inflexible. In order now to enable crease-free stuffing and if appropriate to ensure sufficient shrinkage properties of the skin in the subsequent cooking and scalding process, and also on cooling, which is essential for a smooth, crease-free product, before stuffing the shirred sticks, moistening/soaking is necessary in order to achieve sufficient flexibility and elasticity of the skin (DE-A 3 426 723). This soaking means an additional method step for the processor, which must be avoided for economic reasons.

One solution is that the moistening or soaking step in which the casing material is given the flexibility required for processing is already carried out by the manufacturer and subsequently the moistened material is packaged and delivered. However, this procedure is problematic, since the storage capacity of the casing material for moisture relative to the volume of its packaging for transport and storage is generally low, so that partial drying of the casing material, in particular with moisture loss due to migration from the packaging, can occur. As a result, in turn the service properties important for stuffing such as elasticity and flexibility suffer, so that remoistening by the processor can be necessary.

EP-A 0 248 860 describes that the shirred skins as a result of, for example, differing application of shirring lubricant, possess uneven water absorption during soaking and therefore different elasticities of the skin within the shirred stick occur which at the same time is accompanied by calibre variations during stuffing. For this reason, premoistening of the skin before shirring is carried out in such a manner that the skin is wound up with an internal bubble of water using a moistening machine. This method has the disadvantage that the product cannot be given a uniform moistening over the roll length. The amount of moisture being absorbed by the skin decreases with roll length.

EP-A 0640 289 describes a shirred ready-to-stuff conditioned food casing which, on shirring, is sprayed with an emulsion of water and shirring lubricant, production and uniformity of sprayability being associated with particular difficulties.

EP-A 1 013 173 carries out the moistening process in such a manner that particularly good results are achieved when droplets of a defined size are used.

In the above-described applications, with these skin types the moisture must be applied either at the shirring machine or at the moistening machine. For this, in part difficult special measures must be used. In particular, additional expenditure on measuring must be made.

Because of the permeability of the film pouch, weight losses occur with certain tubular casings which are charged with moisture on packaging the product. These losses can be very considerable, since between packaging and processing, a certain amount of time can pass. With the change of the moisture budget, the service properties also change on stuffing. During stuffing the lower moisture content of the skin becomes very significantly noticeable in the stretching behaviour. In addition, the reliability on sealing, here clipping, decreases markedly owing to the change in moisture state.

In principle, direct addition of water into the transport packaging is also possible. The water would in this case, however, be distributed on the bottom of the packaging, so that it is in direct contact with the skin material. Such shirred sticks with adhering drops of liquid, however, have significantly different stuffing properties during the subsequent stuffing than those without adhering drops of liquid. Exposed adhering drops of liquid in addition involve a high risk of microorganism formation or rapid microbial growth.

However, provision of a uniformly moist climate is desirable not only for packaging water-absorbing food casings, but also for wood processing, for example in the veneer manufacturing sector. There it is necessary, to ensure uniform product quality, that the veneers do not dry out during transport and storage.

### SUMMARY OF THE INVENTION

It was therefore an object of the present invention to provide a regulator which is as technically simple as possible and as inexpensive as possible by which in a space, which is customarily a packaging, a moist climate is produced and it is kept constant over a relatively long period.

Surprisingly, it has now been found that the object can be achieved by a tubular semi-permeable film which is closed on both sides, preferably made of cellulose fibre skin or pure cellulose skin, which is filled with water or a water-swollen absorber and also if appropriate a biocide.

The invention therefore relates to a regulator for providing a climate which is uniformly moist with respect to time and space in a packaging, comprising a tubular semi-permeable film which is closed on both ends and, based on the total weight of the regulator, contains at least 90% by weight, preferably at least 95% by weight, water.

The invention relates to a regulator for providing a climate which is uniformly moist with respect to time and space in a packaging, comprising a tubular semi-permeable film which is closed on both ends and, based on the total weight of the regulator, contains at least 90% by weight water.

The invention also relates to a method for providing a climate which is uniformly moist with respect to time and space in a space which comprises using the inventive regulator.

### DETAIL DESCRIPTION OF THE INVENTION

Semi-permeable with respect to the films relates to the permeability for water or water vapour. Examples of such films are those based on polyamide, cellulose or cellulose fibres.

Preferred semi-permeable films are those based on cellulose or cellulose fibres.

Such regulators according to the invention release, based on the initial weight of the regulator, in the course of 14 days at 20° C., an amount of water of preferably 5 to 20% by weight, particularly preferably 10 to 15% by weight.

Preferably, the water is present in the form of a water-swollen absorber material.

Such an absorber, in the anhydrous state, preferably has a minimum absorption capacity of 20 to 100 g of water per 1 gram of absorber, particularly preferably 40 to 60 g of water per 1 gram of absorber.

Preferably, as such absorbers, use is made of materials based on polyacrylate, silicate or cellulose. Particularly preferably, absorber materials are based on carboxymethylcellulose.

In a preferred embodiment, as water-swollen absorber, use is made of a gel of carboxymethylcellulose having a water fraction of more than 90% by weight.

The moisture regulator has a cylindrical appearance having a diameter D and a length L (both measured in mm). The appearance corresponds to a commercially conventional stuffed sausage. The ratio L to D can be varied in a range from 1.5 to 10, with preferably a ratio of 2.5 to 5 being used. The diameter of the regulator varies according to the amount of the skin material to be moistened and the size of the enclosing carton from 35 mm to 90 mm, preferably 40 to 60 mm.

Preferably, the regulator also comprises an active microbicidal compound with which the tubular semi-permeable film which is closed on both ends is coated or which is present in the interior of the film in the water or the water-swollen absorber.

Preferably, the active compound is present in the interior of the film dissolved in water.

Examples of such microbicidal compounds are potassium sorbate, sodium sulphate, natamycin. A preferred microbicide is potassium sorbate.

Preferably, use is made of 0.5 to 5 g of the abovementioned microbicidal compound per 1 kg of water, particularly preferably, use is made of 1.5 to 2.5 g of the microbicidal compound per 1 kg of water.

As packaging for the shirred sticks, preferably use is made of plastic bags made of polyolefin-containing films, preferably polyethylene-containing films.

The thickness of the films for producing the packaging bags is preferably 20 to 80 μm, particularly preferably 40 to 60 μm.

Such shirred sticks are preferably based on biaxially stretched plastic casings based on polyamide (PA) or artificial skins based on cellulose. Such materials are conventional in the sector of industrial manufacturing of scalded-emulsion and cooked-meat sausage and are described, for example, in DE-A 43 39 337 and EP-A 530 538.

The ratio of the weight of regulator to the weight of shirred sticks is customarily 2 to 12% by weight, preferably 4 to 10% by weight, particularly preferably 4 to 7% by weight.

Particularly preferred ratios result when the regulator has an approximately cylindrical body having a diameter of 50 mm and a length of 200 mm and a weight of 400 g and this is used together with 4 to 6 kg of shirred sticks in a carton volume of 50 to 60 dm³. In this context, carboxymethylcellulose is used as absorber material in the regulator.

By means of the regulators according to the invention, it is possible to provide a constantly moist climate with respect to time and space over a period of 2 to 12 months, preferably 3 to 6 months, on maintaining the above-defined ratio of regulator to the amount of shirred sticks and the volume of the packaging, which is sufficient such that the shirred sticks can be stuffed and further processed immediately after removal from the packaging without further moistening or soaking.

A constantly moist climate is taken to mean a relative humidity of 60 to 95%, preferably 70 to 90%, which is constant over at least 75, preferably 75 to 85, days, i.e. has a deviation of at most 10%, preferably at most 5%.

For mechanical protection, it can be further packaged in surrounding packages, for example made of card or cardboard.

The moisture regulator can be positioned independently of the geometry of the packaging unit. For packaging moisture-sensitive tubular casings, parallelepipedal cartons which are lined internally with a bag made of polyethylene have proven themselves. Within the carton contents, the moisture regulator can adopt all possible positions. The same action for the tubular casings is achieved independently of the position of the moisture regulator (bottom or top).

Preferably, the regulator is used together with a device which prevents the cellulose fibre skin from coming into contact directly with the packaging material to be kept moist. Such a device can be a possibly perforated tube into which the regulator is inserted. Preferably, at least one side of the tube, particularly preferably both sides of the tube, are unsealed.

In a preferred embodiment, the tube consists of 2 half shells connected to one another to form a tube, the two ends of the regulator preferably being fastened between the two half shells connected to one another, for example by clamping. As a result, the regulator hangs semi-free in the device and its moisture-regulating surface has no direct contact with the packaging material to be kept moist and simultaneously has only a minimum interaction with the device.

### EXAMPLES

Calibre constancy: after 2 weeks and after 3 months: Calibre constancy means the geometric uniformity of the product, which was determined via the uniformity of the diameter along a 25 cm long sausage product. Deviations of +/−0.1 mm were rated with a score 1, and deviations of more than 0.5 mm with a score 6.

Stuffing behaviour: after 2 weeks and after 3 months: Stuffing behaviour is taken to mean the reliability with which the skin can be stuffed in the correct manner avoiding bursting of the skin or clips springing off or other faults. A fault-free stuffability of 100 shirred sticks was rated with a score 1. Score 5 was given for 5 faults in 100 shirred sticks. Inability to be stuffed was rated with a score 6.

Hygiene status 14 days after packaging: Hygiene status is taken to mean the microbial loading within the packaging unit. To determine it the shirred sticks were stored in a film bag for 14 days in the presence of the regulator to be tested and then deshirred. The contamination was then investigated using agar plates (Saburaut, type No. 1.10413, Merk, Darmstadt, Germany). For equal to or less than 1 colony-forming unit (CFU) per 100 cm², the score 1 was given, for more than 100 CFU/1 cm², the score 5 was given.

### Example 1

30 m of a polyamide-based, coextruded, biaxially stretched tubular casing (Walsroder® K flex, nominal calibre 60, CaseTech GmbH & Co KG, Walsrode) were shirred on an axial shirring machine having a shirring tube diameter of 42 mm using a shirring lubricant of paraffin oil at an application weight of 1 percent by weight per weight of skin. The resultant shirred stick had a length of 410 mm.

Subsequently, 20 of these shirred sticks were packaged in a carton lined with an LDPE film bag (wall thickness 50 μm) of dimensions length/breadth/height 410 mm/335 mm/335 mm together with a moisture regulator and sealed air-tightly. As regulator, use was made of a cellulose fibre skin closed on both sides with metal clips (Walsroder FR, nominal calibre 50, Casetech GmbH & Co KG, Walsrode) having a weight of 400 g, which was filled with a mixture of water and 1.5 g of potassium sorbate per kg of water.

After storage of 2 weeks of the carton at 20° C. room temperature and 50% relative humidity, the skin was processed without soaking.

### Comparative Example C1

A procedure similar to Example 1 was followed, but addition of a regulator was omitted.

### Comparative Example C2

A procedure as in Example 1 was followed, with here, as regulator, use being made of a tubular film made of nylon 6 having a wall thickness of 30 μm clipped on both ends. Addition of a biocide was omitted.

### Comparative Example 2

A procedure as in Example 1 was followed, but no biocide was added to the regulator.

### Example 3

60 m of a polyamide-based, coextruded, biaxially stretched tubular casing (Walsroder® K flex red, nominal calibre 45, CaseTech, Walsrode, Germany) were spray-moistened with water in addition to the shirring lubricant during shirring on an axial shirring machine having a shirring tube diameter of 40 mm. The resultant shirred sticks had a length of 420 mm. Subsequently, a procedure similar to Example 1 was followed, storage being carried out for 3 months.

### Comparative Example C3

A procedure similar to Example 3 was followed, the use of a moisture regulator being omitted.

The samples from the examples and comparative examples were tested in the application-oriented stuffing experiment using artificial sausage meat emulsion for their suitability for use in practice.

The result of the tests is summarized in the table enclosed as Table 1.

All the references described above are incorporated by reference in its entirety for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

TABLE 1

Samples from example

| | 1 | C1 | C2 | 2 | 3 | C3 |
|---|---|---|---|---|---|---|
| Calibre constancy | 1 | 5 | 2 | 1 | 1 | 3 |
| Stuffing behaviour | 1 | 6 | 2 | 1 | 2 | 4 |
| Hygiene status | 1 | 1 | 4 | 5 | 1 | 1 |

Report scoring system 1 = very good to 6 = very poor

1. A regulator for providing a climate which is uniformly moist with respect to time and space in a packaging, comprising a tubular semi-permeable film which is closed on both ends and, based on the total weight of the regulator, contains at least 90% by weight water.

2. The regulator according to claim 1, wherein, as semi-permeable film, use is made of a film based on cellulose or cellulose fibres.

3. The regulator according to claim 1, wherein the water is present in the form of a water-swollen absorber material.

4. The regulator according to claim 3, wherein said water-swollen absorber material is made of a gel of carboxymethylcellulose having a water fraction of at least 90% by weight.

5. The regulator according to claim 1, wherein the regulator also comprises an active microbicidal compound.

6. The regulator according to claim 5, wherein the active microbicidal compound is potassium sorbate.

7. The regulator according to claim 1, wherein the regulator contains at least 95% by weight water.

8. The regulator according to claim 1, wherein the tubular semi-permeable film closed on both ends contained therein is cylindrical with a length of 40 to 60 mm and a ratio of length L to diameter D of 2.5 to 5.

9. The regulator according to claim 1, which further comprises a perforated tube, the tubular semi-permeable film closed on both ends being arranged therein in such a manner that the moisture-releasing surface is not in direct contact with the inner surface of the tube.

10. A method for providing a climate which is uniformly moist with respect to time and space in a space which comprises using the regulator according to claim 1.

11. The method according to claim 10, wherein the space is a packaging for food casings.

12. The method according to claim 10, wherein the relative humidity achieved therein is 70 to 90% and is constant with a maximum deviation of 5% over at least 75 days.
